(19) United States
(12) Patent Application Publication
(10) Pub. No.: US 2018/0193490 A1
(43) Pub. Date: Jul. 12, 2018

(54) Beta Amyloid Staging

(71) Applicant: GE HEALTHCARE LIMITED, Buckinghamshire (GB)

(72) Inventors: Christopher John Buckley, Buckinghamshire (GB); Adrian Smith, Buckinghamshire (GB)

(73) Assignee: GE HEALTHCARE LIMITED, Buckinghamshire (GB)

(21) Appl. No.: 15/742,512

(22) PCT Filed: Jul. 7, 2016
(86) PCT No.: PCT/EP2016/066196

§ 371 Date: Jan. 7, 2018

(30) Foreign Application Priority Data
Jul. 7, 2015 (GB) 1511846.6

(51) Int. Cl. A61K 51/04

(52) U.S. Cl. CPC A61K 51/04; A61K 51/0455; A61K 51/0453

(57) ABSTRACT

The present invention relates to a method of in vivo imaging and in particular to a method for the evaluation of in vivo images of beta amyloid (Aβ) distribution in the brain of a subject suspected of having Alzheimer's disease (AD). The method of the present invention provides more detailed information to the clinician as compared with prior art methods, facilitating identification of those subjects who will benefit most from disease modifying therapies.

### TECHNICAL FIELD OF THE INVENTION

The present invention relates to in vivo imaging and in particular to in vivo imaging of beta amyloid (Aβ) plaques in the brain of a subject. Methods are provided herein for objective determination of the stage of Aβ pathology in a subject.

### DESCRIPTION OF RELATED ART

Amyloid is an abnormal deposit of insoluble protein fibrils in a body tissue or organ. It is characterised by unique staining properties, electron microscopic appearance, and a β-pleated sheet pattern on X-ray diffraction analysis. Amyloid can be formed from a selection of at least 18 proteins, and it can accumulate in tissue to form visible plaques. It is associated with over 30 human diseases, most notably Alzheimer's disease (AD). The specific type of amyloid involved in AD is beta amyloid (Aβ), which is the main component of Aβ plaques (can also referred to as neuritic plaquies). Aβ is one of the two neuropathological hallmarks of AD that can be seen microscopically in brain tissue specimens stained with certain dyes, the other being neurofibrillary tangles (NFT) of Tau protein. Aβ is a protein fragment snipped from an amyloid precursor protein (APP). In healthy brain, these protein fragments are broken down and eliminated, whereas in AD the fragments accumulate to form hard, insoluble plaques. NFT are insoluble twisted fibres found inside brain cells and consist primarily of the protein Tau. Tau forms part of a structure called a microtubule that helps to transport nutrients and other important substances from one part of the nerve cell to another. In AD the Tau protein is abnormal and the microtubule structures collapse. Definitive diagnosis of AD is carried out by examination of brain tissue at autopsy for the presence of Aβ plaques and NFT.

A number of positron emission tomography (PET) imaging agents that bind to Aβ are commercially-available: Florbetaben F-18 (Piramal Imaging/Neuraceq™), Florbetapir F-18 (Lilly/Amyvid™), and Flutemetamol F-18 (GE Healthcare/Vizamyl™). These PET imaging agents enable detection in living subjects of Aβ build up in plaques and in the blood vessels supplying the brain. A positive Aβ PET scan on its own is not definitive for AD but rather is a diagnostic tool that facilitates determination of whether there is Aβ in the brain, increasing the clinical certainty of diagnosis during life. Other Aβ imaging agents are in clinical development, e.g. Navidea's NAV4694 compound.

A negative scan using an Aβ PET imaging agent is regarded as one where there is normal uptake in the cortical grey matter and good grey-white matter contrast. A negative scan indicates few to no Aβ plaques. If there is cognitive impairment in conjunction with a negative scan, the cause is likely to be something other than AD.

A positive scan using an Aβ PET imaging agent is regarded as one where there is increased uptake in cortical grey matter and a loss of grey-white matter contrast. A positive scan indicates moderate to frequent plaques, which may be found in patients with AD, but also in patients with other types of cognitive impairment and in older people with normal cognition.

Thal et al (2002 Neurology; 58: 1791-1800) propose five phases of Aβ amyloidosis (“Thal Phases”) based on histopathological assessment of post-mortem brain tissue. Thal Phases 1-5 can be summarised as follows:
1 Aβ deposits are found exclusively in the neocortex.
2 Additional involvement of allocortical brain regions.
3 Diencephalic nuclei, the striatum, and the cholinergic nuclei of the basal forebrain exhibit Aβ deposits.
4 Several brainstem nuclei become additionally involved.
5 Cerebellar Aβ deposition.

Thal et al (supra) noted that the phases of Aβ amyloidosis correlated significantly with the evolution of neurofibrillary lesions and also that 17 clinically-proven AD cases exhibited Aβ phases 3, 4, or 5, whereas 9 nondemented cases with AD-related Aβ pathology showed Aβ phases 1, 2, or 3. These observations suggest that the Thal Phases 1-3 correlate with pre-clinical AD.

It is still required for a diagnosis of AD to have measurements of Tau as well as Aβ so that, taken alone, the current Aβ in vivo imaging methods are not sufficient to reach a definitive diagnosis of AD. There is therefore scope for improved in vivo imaging methods in the diagnosis of AD in living subjects.

### SUMMARY OF THE INVENTION

In one aspect, the present invention provides a method for staging beta amyloid (Aβ) pathology in a subject's brain wherein said method comprises:
(i) obtaining an in vivo image of said subject's brain using a Aβ imaging agent;
(ii) determining from said in vivo image the uptake of said Aβ imaging agent in a cortical region of said brain; and,
(iii) determining from said in vivo image the uptake of said Aβ imaging agent in a striatal region of said brain;
   wherein positive uptake of said Aβ imaging agent in said cortical region and negative uptake of said Aβ imaging agent in said striatal region indicates Thal Phase 3 Aβ pathology;
   and wherein positive uptake of said Aβ imaging agent in said cortical region and positive uptake of said Aβ imaging agent in said striatal region indicates Thal Phase 4 or 5 Aβ pathology.

In another aspect, the present invention provides a method for treatment of Alzheimer's disease (AD) wherein said method comprises the method for staging Aβ pathology of the invention and the further steps of:
(iv) selecting those subjects in whom Thal Phase 3 Aβ pathology or greater has been indicated;
(v) treating those subjects selected in step (iii) with an AD therapy.

In another aspect, the present invention provides a method for the evaluation of the effects of an experimental AD therapy wherein said method comprises carrying out the method for staging Aβ pathology of the invention on a group of subjects to whom said experimental AD therapy has been given.

Currently, a typical Aβ PET assessment considers uptake of the imaging agent in either the cortex or the striatum as indicative of an abnormal scan. In contrast, the present invention considers uptake in the cortex and striatum separately. Using the method of the invention those subjects that have a cortical Aβ burden but minimal to no striatal Aβ burden on an in vivo image can be identified as those whose brain Aβ levels have not progressed to advanced AD. Those subjects who are showing cortical Aβ but not striatal Aβ on an in vivo image are likely to be among those who will benefit most from disease modifying therapies. The present invention therefore makes use of a more specific interpretation of Aβ in vivo images that provides useful additional information to the clinician as compared with prior art methods.

### BRIEF DESCRIPTION OF THE FIGURES

FIG. 1 illustrates the algorithm applied for the statically-determined thresholds.

### DETAILED DESCRIPTION OF THE PREFERRED EMBODIMENTS

To more clearly and concisely describe and point out the subject matter of the claimed invention, definitions are provided hereinbelow for specific terms used throughout the present specification and claims. Any exemplification of specific terms herein should be considered as a non-limiting example.

The terms “comprising” or “comprises” have their conventional meaning throughout this application and imply that the agent or composition must have the essential features or components listed, but that others may be present in addition. The term ‘comprising’ includes as a preferred subset “consisting essentially of” which means that the composition has the components listed without other features or components being present.

The term “staging” refers to the process of determining the extent to which a disease has developed.

The term “Aβ pathology” refers to the progression of Aβ deposition in the brain of a subject.

The “subject” of the invention is a living human or animal subject. In one embodiment the subject of the invention is a primate from the family Hominidae (also known as great apes). In one embodiment the subject of the invention is a human. In one embodiment said subject is suspected of having Alzheimer's disease.

The step of “obtaining an in vivo image” comprises carrying out an in vivo imaging procedure on the subject of the invention using a Aβ imaging agent. Methods of in vivo imaging are known to those of skill in the art as described for example in “Textbook of in vivo Imaging in Vertebrates” (2007 Wiley; Vasilis Ntziachristos, Anne Leroy-Willig, Bertrand Tavitian, Eds.) and in “Handbook of Radiopharmaceuticals” (2003 Wiley; Michael J Welch and Carol S Redvanly, Eds.). An exemplary in vivo imaging procedure suitable for the present invention comprises parenteral administration of the Aβ imaging agent to a subject followed by detecting the distribution of uptake of said Aβ imaging agent in said subject after a defined period of time using an in vivo imaging apparatus to produce an image of said distribution.

The term “Aβ imaging agent” refers to any in vivo imaging agent that binds to Aβ with high affinity and has a good brain pharmacokinetic profile. In one embodiment said Aβ imaging agent comprises a radiolabelled compound.

In one embodiment said Aβ imaging agent is either a positron emission tomography (PET) imaging agent or a single photon emission tomography (SPECT) imaging agent.

In one embodiment said Aβ imaging agent is a PET imaging agent.

In one embodiment said PET imaging agent comprises a compound radiolabelled with ¹¹C or ¹⁸F.

In one embodiment said PET imaging agent comprises a compound radiolabelled with ¹¹C.

In one embodiment said PET imaging agent is ¹¹C-PIB:

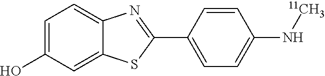 [SMILES: [11CH3]Nc1ccc(-c2nc3ccc(O)cc3s2)cc1]

In one embodiment said PET imaging agent comprises a compound radiolabelled with ¹⁸F.

In one embodiment said PET imaging agent is selected from one of the following compounds:

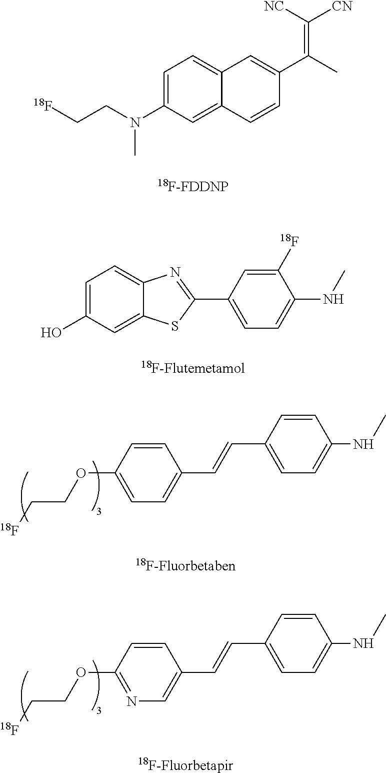

In one embodiment said PET imaging agent is ¹⁸F-Flutemetamol.

In one embodiment said PET imaging agent is ¹⁸F-Fluorbetapir.

In one embodiment said Aβ imaging agent is a SPECT imaging agent.

In one embodiment said SPECT imaging agent comprises a compound radiolabelled with ¹²³I or ¹²⁵I.

In one embodiment said SPECT imaging agent is:

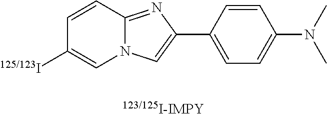

The step of “determining the uptake of said Aβ imaging agent” from said in vivo image is carried out by visually inspecting the in vivo image. In one embodiment said visual inspection is facilitated by software and carried out using an in vivo image on an electronic screen. An illustrative non-limiting example of how this is done in the context of the present invention is as follows:
Display the image with all planes (axial, sagittal and coronal planes) linked by crosshairs.
Select a colour scale that provides a progression of low through high intensity (e.g. rainbow, spectrum or Sokoloff). The selected colour scale should (1) provide colours that allow the reader to discriminate intensity levels above and below the intensity level of the pons, (2) provide a colour for regions with little or no Aβ binding such as the cerebellar cortex, and (3) provide a range of distinct colours above 50 to 60% of the peak intensity.
Display the reference scale. Adjust the colour scale to set the pons to approximately 90% maximum intensity.

In one embodiment the Aβ imaging agent is ¹⁸F-Flutemetamol (Vizamyl™) and the determination of its uptake is carried out according to the FDA prescribing information at this link:
http://www.accessdata.fda.gov/druqsatfda_docs/label/2014/203137s002lbl.pdf.

A “cortical region” is defined herein as any part of the cortex, which is the outer layer of neural tissue in the brain of the subject of the invention. The cortical region is grey matter, consisting mainly of cell bodies (with astrocytes being the most abundant cell type in the cortex as well as the human brain as a whole) and capillaries. It contrasts with the underlying white matter, consisting mainly of the white myelinated sheaths of neuronal axons.

A “striatal region” is defined herein as any part of the striatum, which is the subcortical part of the forebrain. The striatum receives input from the cortex and is the primary input to the basal ganglia system of the brain. In all primates, the striatum is divided by a white matter tract called the internal capsule into two sectors called the caudate nucleus and the putamen.

In one embodiment uptake of said Aβ imaging agent in any one of the frontal/anterior cingulate, the posterior cingulate/precuneus, the insula, the inferior parietal and the lateral temporal lobe is taken to indicate uptake in said cortical region.

In one embodiment uptake of said Aβ imaging agent at level of the head of the caudate nucleus and putamen is taken to indicate uptake in said striatal region.

Whether uptake in either the cortical region or the striatal region is “positive” or “negative” can be determined by visual assessment by a trained reader against specified criteria, i.e. dichotomy as “positive” or “negative”. The term “positive” can also be understood more generally to refer to relatively high uptake of said Aβ imaging agent and the term “negative” to refer to relatively low uptake of said Aβ imaging agent.

In one embodiment a cortical negative scan has the following characteristics:
frontal, lateral temporal, inferolateral parietal lobes: gradual gradient from bright intensity of the white matter to lower intensity at the periphery of the brain; distinct sulci with concave surfaces (white matter sulcal pattern); and,
posterior cingulate and precuneus: grey matter uptake below 50-60% (according to a scheme where the pons has been set as 90% as described above) of peak intensity; gap of lower intensity separates two hemispheres on coronal view.

In one embodiment cortical positive can be understood to be where at least one cortical region has a reduction or loss of the normally distinct grey-white matter contrast. These scans have one or more regions with increased cortical grey matter signal (above 50-60% peak intensity) and/or reduced (or absent) grey/white matter contrast (white matter sulcal pattern is less distinct). A positive scan may have one or more regions in which grey matter radioactivity is as intense or exceeds the intensity in adjacent white matter.

In one embodiment a cortical positive scan has the following characteristics:
frontal, lateral temporal, or inferolateral parietal lobes: high intensity seen to the periphery of the brain, with sharp reduction of intensity at the brain margin; sulci not distinct due to fill-in by high intensity grey matter resulting in a convex surface at the edge of the brain; or,
posterior cingulate and precuneus: grey matter uptake above 50-60% of peak intensity; high grey matter intensity that closes the gap between the two hemispheres on coronal view.

In one embodiment a striatal negative scan would have the following characteristics:
approximately 50% of peak intensity or lower in the region between the higher intensities of the thalamus and frontal white matter (striatal “gap”).

In one embodiment a striatal positive scan would have the following characteristics:
intensity above 50-60% of peak intensity in the region between the thalamus and frontal white matter (striatal “gap”); gap between thalamus and frontal white matter not distinct.

In an alternative embodiment whether a region is positive or negative can be assessed by measured determination of uptake by standardised uptake value ratio (SUVR) above a predetermined threshold—a continuous variable; the ratio of standard uptake of the region of interest (ROI) is divided by the standard uptake value of a reference region. The “region of interest” is the anatomic region for which the SUVR measure is desired. In the context of the present invention the region of interest would be: for cortical assessment a grey matter cortical volume within the cortex (frontal lobe, inferior parietal lobe, lateral temporal lobe or posterior cingulate/precuneus or similar), and for striatal assessment a subcortical volume within the putamen/caudate nucleus The “reference region” is a non-cortical/non-striatal region of the brain of the subject in which the uptake is used as the denominator to normal uptake across regions of interest. The “threshold” for SUVR positivity varies according to the reference regions used and the geometric configuration of the region of interest. In this embodiment a cortical negative scan would have SUVR for all cortical regions assessed equal to or below predetermined thresholds, a cortical positive scan would have SUVR for any cortical region assessed above predetermined thresholds, a striatal negative scan would have striatal SUVR is assessed as equal to or below the predetermined threshold and a striatal positive scan would have striatal SUVR assessed as above the predetermined threshold.

The term “Thal Phase 3 Aβ pathology or greater” refers to any one of Thal Phase 3, 4 or 5. In one embodiment of the method of treatment of the present invention said Thal Phase 3 Aβ pathology or greater is Thal Phase 3. In one embodiment of the method of treatment of the present invention said Thal Phase 3 Aβ pathology or greater is Thal Phase 4. In one embodiment of the method of treatment of the present invention said Thal Phase 3 Aβ pathology or greater is Thal Phase 5.

Amyloid imaging is a helpful diagnostic tool and in one embodiment the present invention can serve as a secondary outcome measure in AD clinical trials with disease-modifying agents. Non-limiting examples of such disease-modifying agents include the anti-amyloid monoclonal antibodies bapineuzumab and solanezumab (Rinne et al 2010 Lancet Neurol; 5: 363-372; Farlow et al 2012 Alzheimers Demen; 5: 261-271). In particular, the potential provided by the method of the present invention to identify early stages of AD may assist in the recruitment of subjects whose disease is progressing (Jack et al., 2013 Lancet Neurology 12: 207-216; Villemagne et al., 2013 Lancet Neurology 12: 357-367) and who may benefit most from disease modifying treatments to which advanced disease may be refractory (Salloway et al, 2014 NEJM 370(4): 322-333) and Salloway et al 2014 NEJM 370(15): 1459-1460.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties, as if they were individually incorporated.

### Brief Description of the Examples

Example 1 presents an analysis of separate cortical and striatal determinations of in vivo images obtained with the Aβ imaging agent Flutemetamol.

### List of Abbreviations Used in the Examples

Aβ amyloid beta
BIE(s) blinded image evaluation(s)
BSS Bielschowsky silver stain
C−S− cortical negative striatal negative
C+S− cortical positive striatal negative
C+S+ cortical positive striatal positive
C−S+ cortical negative striatal positive
CTX cortex
IHC immunohistochemistry
PET positron emission tomography
ROC receiver operating characteristic
SD standard deviation
STR striatal
SUVR standardised uptake value ratio
VOI volume of interest

### EXAMPLES

### Example 1: Analysis of Separate Cortical and Striatal Determinations

The data presented explore the use of separate cortical and striatal assessments to aid the Thal (amyloid) phase assignation to in life subjects. The data sets used are from a Flutemetamol (18F) Injection Phase III clinical trial.

In this trial PET image assessment was compared to histopathological assessment of amyloid pathology from autopsy.

The available data for a preliminary analysis of amyloidosis staging using separate cortical and striatal assessments comes from two sources:
(1) The blinded image evaluations (BIEs) from the GE067-026 clinical trial (NCT02090855)
(2) Provisional SUVR determinations in post-hoc analyses of the GE067-026 data

### GE067-026 BIE Analysis

During the BIEs for the above trial, 5 independent readers assessed each of 106 amyloid PET images as abnormal or normal. These dichotomous assessments formed the primary analysis for the trial. However, the individual cortical and striatal assessments were recorded and were re-analysed to estimate the cortical and striatal status separately.

For each subject, each of the five readers rated 4 cortical regions and 1 striatal region as Aβ positive or Aβ negative.¹ ¹ All regions assessed are bilateral. Readers assessed images bilaterally—left and right hemispheres—but recoded only one result for each of the 5 bilateral regions.

Applying the same general principle that if any of the 4 cortical regions are positive this confers cortical positivity, separate cortical and striatal assessments were determined for each case.

A summary (Table 1) shows that one reader (Reader D) consistently underscored the striatal positivity.

TABLE 1
Subject classification of the GE067-026
data set by BIE assessment (N = 106)

| | Reader A | Reader B | Reader C | Reader D | Reader E |
|---|---|---|---|---|---|
| C−S− | 28 | 35 | 37 | 34 | 30 |
| C+S− | 5 | 4 | 6 | 38 | 5 |
| C−S+ | 1 | 0 | 0 | 0 | 2 |
| C+S+ | 73 | 68 | 64 | 35 | 70 |

Striatal PET positivity is usually associated with frequent striatal plaques (see Table 2)

TABLE 2
BIE striatal assessment against striatal plaque count
by histopathology (N = 108 subjects and 5 readers)

| STR Plaques | None | Sparse | Moderate | Frequent |
|---|---|---|---|---|
| C−S− | 98 | 19 | 24 | 23 |
| C+S− | 1 | 1 | 17 | 37 |
| C+S+ | 0 | 0 | 18 | 289 |

By Majority assessment 6 cases were identified as C+S− (Table 3) 4 (75%) of which were phase 3 and 2 were phase 4.

TABLE 3
BIE majority categorisation by Thal (amyloid phase)

| Maj | C−S− | C+S− | C+S+ | C−S+ |
|---|---|---|---|---|
| Phase 0 | 7 | 0 | 0 | 0 |
| Phase 1 | 10 | 0 | 0 | 0 |
| Phase 2 | 5 | 0 | 0 | 0 |
| Phase 3 | 7 | 4 | 3 | 0 |
| Phase 4 | 5 | 2 | 17 | 0 |
| Phase 5 | 0 | 0 | 46 | 0 |

### Provisional SUVR Analysis

Some work has been performed to assess the quantitative SUVR measures from the GE067-026 cohort.

In an automated SUVR assessment methodology using GE software Cortex ID (J Lilja) PET images were quantitated using separate cortex and striatal VOIs. The SUVRs were determined for the cortical and striatal values using 3 different reference regions; whole cerebellum, cerebellar grey and pons.

Thresholding was determined by multiple methods to identify which method provided the greatest accuracy (See Setting SUVR thresholds)

The thresholds determined by ROC analysis for Phase 3 or above being abnormal gave the best sensitivity and specificity for cortical assessments. A similar approach gave the best sensitivity and specificity for the striatal assessments. Using the whole cerebellum as a reference region or pons as the reference region gave the best sensitivity and specificity (see Table 4).

Combining these thresholds to determine cortical and striatal positivity based upon SUVRₚₒₙₛ values gave similar results to those see by BIE assessment. Of 5 C+S− cases 3 (66%) were Thal phase 3.

Interestingly, however, of the 3 Thal phase 3 C+S− cases determined by SUVR analysis, only two were in common with those identified by BIE assessment and so BIE and SUVR analysis may be complementary methods to identify C+S− Phase 3 cases; between the two methods 6 phase 3 cases were identified as C+S−.

TABLE 4
SUVR Analysis of Cortical and Striatal Beta Amyloid Uptake

| Amyloid Thal phase | C−S− | C+S− | C+S+ | C−S+ |
|---|---|---|---|---|
| 0 | 7 | 0 | 0 | 0 |
| 1 | 8 | 0 | 0 | 0 |
| 2 | 5 | 0 | 0 | 0 |
| 3 | 8 | 3 | 1 | 0 |
| 4 | 3 | 1 | 16 | 2 |
| 5 | 1 | 1 | 44 | 0 |

### 1.1 Setting SUVR Thresholds

SUVR thresholds were set using two methods.
(1) Statistical determination
(2) Receiver operator characteristics

Other methods of threshold determination could be considered

### (1) Statistical Determination

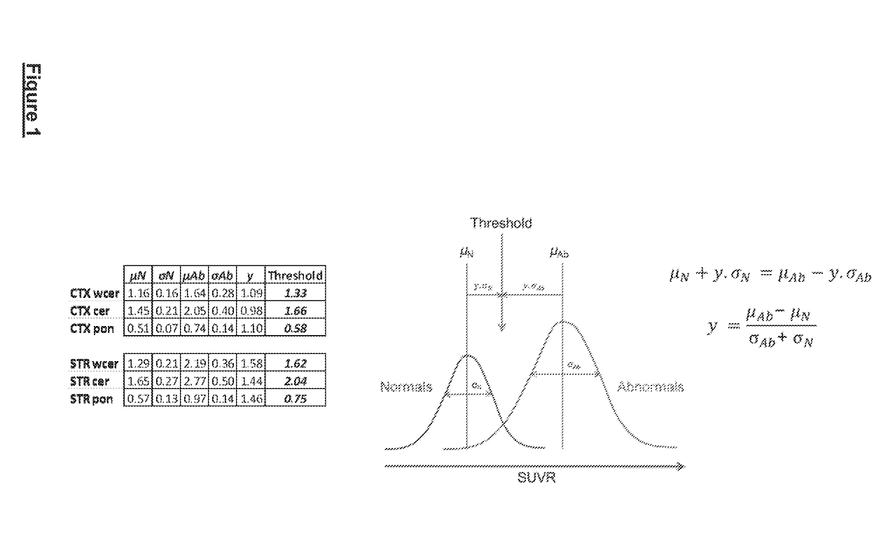

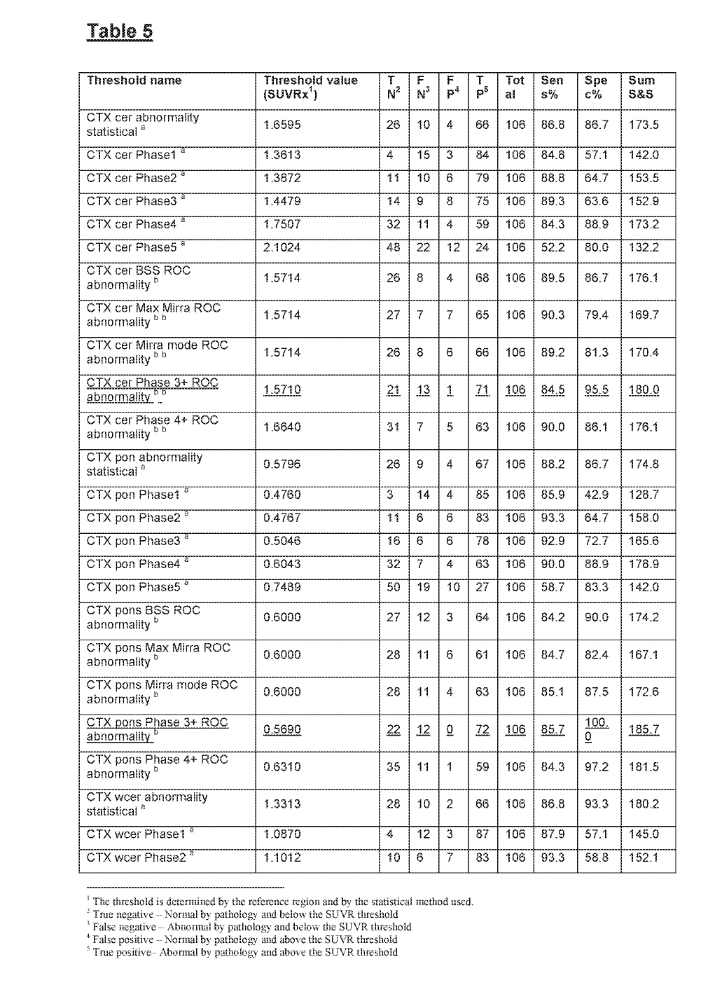

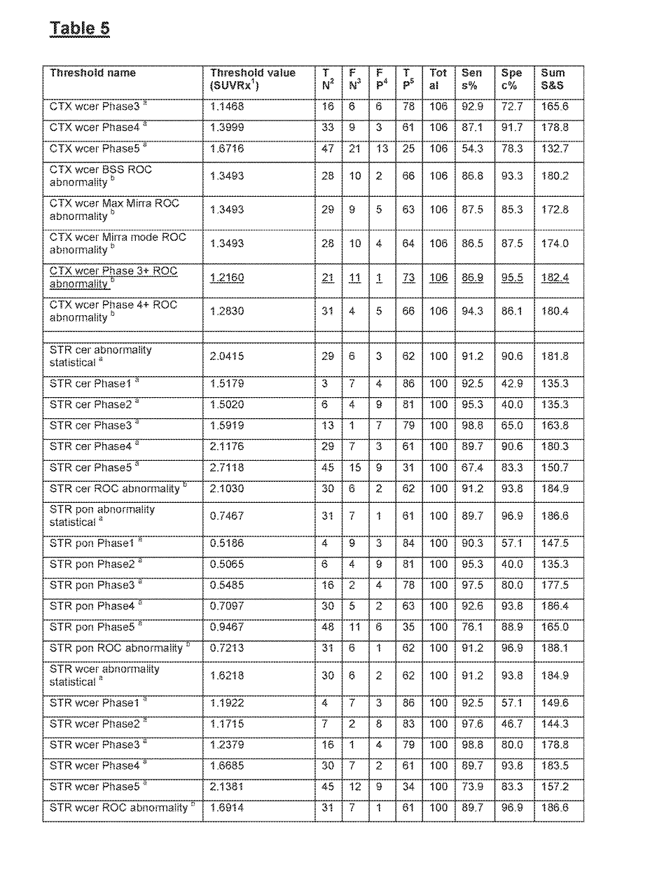

For statically determined thresholds the algorithm depicted in FIG. 1 was applied. Briefly, all cases were identified as negative or positive based on histopathology. For the cortex this was based on the GE067-026 standard of truth²—abnormal or normal based on any regional Bielschowsky score>1.5=abnormal and all regional scores≤1.5=normal. For the striatum this was based on the identification of “Frequent” plaques by amyloid-β IHC (4G8). The threshold determinations are calculated in the file Cortex ID SUVR thresholds.xlsx. ² For the GE067-007 and -026 autopsy trials the primary endpoint was Bielschowsky silver stain (BSS) scores of CERAD based neuritic plaque density. 0=none, 1=sparse, 2=moderate and 3=frequent. Multiple measures were averaged and the arithmetic mean between none/sparse (normal) and moderate/frequent (abnormal=1.5. Any case with a regional BSS score>1.5 was deemed abnormal.

The SUVR means and standard deviations were calculated for normal and abnormal subjects for each of the SUVR measures; Cortex and striatum and for each of the SUVR reference regions. The threshold was determined to be the SUVR value at which the fractional standard deviation is equal between the two population means i.e. when the normal mean plus y times the normal SD is equal to the abnormal mean minus y times the abnormal SD.

### Dichotomous Threshold

A statistically based threshold was determined for abnormal and normal cases (based on histopathology) and applied to cortical and striatal SUVRs separately.

### Staging Threshold

A variant of the above threshold was determined based upon the Thal phases. Thus the threshold for the cortex and striatum was determined between phase 0 and 1, between phase 1 and phase 2 . . . etc. This gave 15 threshold values (5 to discriminate each phase and 1 for each of the 3 reference regions; 15 in total)

FIG. 1 illustrates the cortex and striatal SUVR thresholds and calculation method.

For the GE067-007 (NCT01165554) and -026 autopsy trials the primary endpoint was Bielschowsky silver stain (BSS) scores of CERAD based neuritic plaque density. 0=none, 1=sparse, 2=moderate and 3=frequent. Multiple measures were averaged and the arithmetic mean between none/sparse (normal) and moderate/frequent (abnormal=1.5. Any case with a regional BSS score>1.5 was deemed abnormal.

### (2) Receiver Operator Characteristics

This is an empirically determined threshold that by definition identifies the threshold that gives the best sensitivity and specificity.

Receiver operator characteristics analysis determines the sensitivity and specificity given a variable threshold. The most appropriate threshold is then determined by the maximum of the sum of the sensitivity and specificity.

Table 5 (presented in the figures section) shows the threshold values determined for the preliminary SUVR analyses (“a” refers to a statistically-determined threshold and “b” to a receiver operator characteristic threshold.

Table 6 shows the sums of sensitivity and specificity by threshold and reference region. The data shows the superiority of wcer and pons as reference regions and ROC analysis using phase 3 or above as the thresholding criterion.

TABLE 6

| Threshold reference region | Wcer | Cer | Pons | Average |
|---|---|---|---|---|
| CTX abnormality statistical threshold | 180% | 174% | 175% | 176% |
| CTX Phase1 threshold | 145% | 142% | 129% | 139% |
| CTX Phase2 threshold | 152% | 153% | 158% | 155% |
| CTX Phase3 threshold | 166% | 153% | 166% | 161% |
| CTX Phase4 threshold | 179% | 173% | 179% | 177% |
| CTX Phase5 threshold | 133% | 132% | 142% | 136% |
| CTX BSS ROC abnormality threshold | 180% | 176% | 174% | 177% |
| CTX Max Mirra ROC abnormality | 173% | 170% | 167% | 170% |
| threshold | | | | |
| CTX Mirra mode ROC abnormality | 174% | 170% | 173% | 172% |
| threshold | | | | |
| CTX Phase 3 + ROC abnormality | 182% | 180% | 186% | 183% |
| threshold | | | | |
| CTX Phase 4 + ROC abnormality | 180% | 176% | 182% | 179% |
| threshold | | | | |
| STR abnormality statistical threshold | 185% | 182% | 187% | 184% |
| STR Phase1 threshold | 150% | 135% | 147% | 144% |
| STR Phase2 threshold | 144% | 135% | 135% | 138% |
| STR Phase3 threshold | 179% | 164% | 178% | 173% |
| STR Phase4 threshold | 183% | 180% | 186% | 183% |
| STR Phase5 threshold | 157% | 151% | 165% | 158% |
| STR ROC abnormality threshold | 187% | 185% | 188% | 187% |
| Average | 168% | 163% | 168% | |

1. A method for staging beta amyloid (Aβ) pathology in a subject's brain wherein said method comprises:
(i) obtaining an in vivo image of said subject's brain using a Aβ imaging agent;
(ii) determining from said in vivo image the uptake of said Aβ imaging agent in a cortical region of said brain; and,
(iii) determining from said in vivo image the uptake of said Aβ imaging agent in a striatal region of said brain;
wherein positive uptake of said Aβ imaging agent in said cortical region and negative uptake of said Aβ imaging agent in said striatal region indicates Thal Phase 3 Aβ pathology
and wherein positive uptake of said Aβ imaging agent in said cortical region and positive uptake of said Aβ imaging agent in said striatal region indicates Thal Phase 4 or 5 Aβ pathology.

2. The method as defined in claim 1 wherein said subject is a mammalian subject.

3. The method as defined in claim 1 wherein said subject is a human subject.

4. The method as defined in claim 3 wherein said subject is suspected of having Alzheimer's disease.

5. The method as defined in claim 1 wherein uptake of said Aβ imaging agent in any one of the frontal/anterior cingulate, the posterior cingulate/precuneus, the insula and the lateral temporal lobe is taken to indicate uptake in said cortical region.

6. The method as defined in claim 1 wherein uptake of said Aβ imaging agent at level of the head of the caudate nucleus and putamen is taken to indicate uptake in said striatal region.

7. The method as defined in claim 1 wherein said Aβ imaging agent is either a positron emission tomography (PET) imaging agent or a single photon emission tomography (SPECT) imaging agent.

8. The method as defined in claim 1 wherein said Aβ imaging agent is a PET imaging agent.

9. The method as defined in claim 8 wherein said PET imaging agent comprises a compound radiolabelled with ¹¹C or ¹⁸F.

10. The method as defined in claim 9 wherein said PET imaging agent comprises a compound radiolabelled with ¹¹C.

11. The method as defined in claim 10 wherein said PET imaging agent is ¹¹C-PIB:

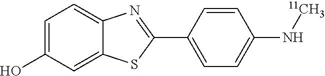 [SMILES: [11CH3]Nc1ccc(-c2nc3ccc(O)cc3s2)cc1]

12. The method as defined in claim 9 wherein said PET imaging agent comprises a compound radiolabelled with ¹⁸F.

13. The method as defined in claim 12 wherein said PET imaging agent is selected from one of the following compounds:

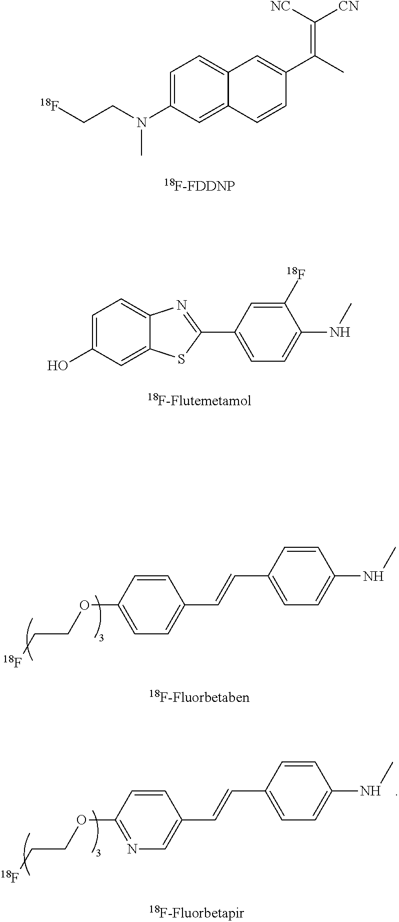

14. The method as defined in claim 1 wherein said Aβ imaging agent is a SPECT imaging agent.

15. The method as defined in claim 14 wherein said SPECT imaging agent comprises a compound radiolabelled with ¹²³I or ¹²⁵I.

16. The method as defined in claim 15 wherein said SPECT imaging agent is:

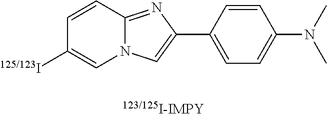

17. A method for treatment of Alzheimer's disease (AD) wherein said method comprises the method as defined in claim 1 and the further steps of:
(iv) selecting those subjects in whom Thal Phase 3 Aβ pathology or greater has been indicated;
(v) treating those subjects selected in step (iii) with an AD therapy.

18. A method for the evaluation of the effects of an experimental AD therapy wherein said method comprises carrying out the method as defined in claim 1 on a group of subjects to whom said experimental AD therapy has been given.

19. A method to determine inclusion of subjects into a clinical trial for the evaluation of an Aβ imaging agent comprising the method as defined in claim 1 and admitting those subjects having positive uptake of said Aβ imaging agent in said cortical region and negative uptake of said Aβ imaging agent in said striatal region into said clinical trial.
